(19) United States
(12) Patent Application Publication
(10) Pub. No.: US 2026/0083790 A1
(43) Pub. Date: Mar. 26, 2026

(54) Decarboxylated Cannabis Pill and Method of Producing the Pill

(71) Applicant: Terry Teruya, Honolulu, HI (US)

(72) Inventor: Terry Teruya, Honolulu, HI (US)

(21) Appl. No.: 19/338,449

(22) Filed: Sep. 24, 2025

Related U.S. Application Data
(60) Provisional application No. 63/698,652, filed on Sep. 25, 2024.

(51) Int. Cl. A61K 36/185; A61K 9/48; A61K 31/00; A61K 31/375; A61K 31/593; A61K 33/06; A61K 33/26; A61K 33/30

(52) U.S. Cl. CPC A61K 36/3482; A61K 9/4825; A61K 9/4833; A61K 9/4858; A61K 9/4891; A61K 31/375; A61K 31/593; A61K 31/658; A61K 33/06; A61K 33/26; A61K 33/30; A61K 2236/17; A61K 2236/37; A61K 2236/50

(57) ABSTRACT

An orally administered cannabis pill tailored for medicinal and recreational applications and consumption is disclosed. The pill contains decarboxylated cannabis and in some embodiments can include flavoring agents, mineral and vitamin supplements, and Lecithin. The pill is in the form of a gelatin capsule and disintegrates within the human body for providing cannabis advantages without affecting lung health. The invention also includes a method for producing the pill and includes sourcing of fresh and organic cannabis flowers containing cannabinoids. The flowers are subjected to heat and pressure to extract essential oils and produce concentrated cannabis extract discs. Decarboxylation is done to convert THCA into psychoactive THC. Subsequently, the decarboxylated cannabis extract is finely ground into a powder and encapsulated within size 00 gelatin capsules.

### CROSS-REFERENCE TO RELATED APPLICATION

The present application claims priority to, and the benefit of, U.S. Provisional Application No. 63/698,652, which was filed on Sep. 25, 2024, and is incorporated herein by reference in its entirety.

### FIELD OF THE INVENTION

The present invention generally relates to the field of providing medicinal and recreational effects of cannabis. More specifically, the present invention relates to a novel orally administered cannabis pill that contains decarboxylated cannabis included in a gelatin body. The pill obviates smoking cannabis and enables users to obtain pain and stress relief through cannabis with a simple pill. Accordingly, the present disclosure makes specific reference thereto. Nonetheless, it is to be appreciated that aspects of the present invention are also equally applicable to other like applications, devices, and methods of manufacture.

### BACKGROUND

By way of background, Cannabis is a genus of the flowering plant Cannabis and is used for medicinal purposes, as a recreational drug, and is also used for different industrial purposes. Cannabis is typically administered via inhalation such as through smoking or vaping or consumption of marijuana-infused food and drink. Cannabis plants produce a group of chemicals called cannabinoids, which produce mental and physical effects when consumed. Cannabinoids are a group of 21-carbon-containing terpenophenolic compounds produced by Cannabis species.

Specially, cannabis provides substantial benefits to patients suffering from a wide range of symptoms experienced in connection with various medical conditions. Cannabis provides relief from chronic pain such as pain caused from spinal cord injury, HIV or complex regional pain syndrome, cancer, chemotherapy, muscle and joint problems, rheumatoid arthritis, multiple sclerosis, and more.

When cannabis is smoked, Cannabis smoke, like tobacco smoke, irritates the airways, causes inflammation, and can lead to symptoms like chronic cough, wheezing, and phlegm production. Repeated inflammation can damage lung tissue and cause scarring, which can permanently reduce lung function and airflow. Cannabis smoke can also damage the cilia in the airways which helps clear mucus and debris. This can increase the risk of infections. Health professionals advise against smoking cannabis, especially for individuals with pre-existing lung conditions. Often, smoking cannabis does not provide or administer a required quantity of cannabis and desired effect. As a result, users end up smoking either less or more cannabis across multiple servings. Also, cannabis edibles such as gummies and candies have sugar and high calories. Diabetic and other health-conscious people avoid having such cannabis edibles. People desire improved ways of ingesting cannabis orally as an alternative to smoking for preventing disadvantages associated with cannabis smoke.

Therefore, there exists a long-felt need in the art for ingesting cannabis orally, typically in the form of pills such as capsules. Additionally, there is a long-felt need in the art for a cannabis pill that obviates smoking cannabis for recreational and medicinal benefits of cannabis. Moreover, there is a long-felt need in the art for an orally administered cannabis pill to help alleviate chronic pain and providing recreational effect of the cannabis. Further, there is a long-felt need in the art for a cannabis medication pill that uses a gelatin capsule with decarboxylated cannabis for using cannabis for medicinal and recreational purposes. Furthermore, there is a long-felt need in the art for an oral cannabis pill that provides an adequate and precise dose of cannabis for medicinal or recreational purposes. Also, there is a long-felt need in the art for a cannabis pill that enables users to obtain pain and stress relief through cannabis while orally ingesting the pill without the negative effects of high calorie edibles. Finally, there is a long-felt need in the art for a decarboxylated cannabis capsule or pill that provides an optimal or measured effect of cannabis without side effects of cannabis smoke and is safe for the lungs of users.

The subject matter disclosed and claimed herein, in one embodiment thereof, comprises an orally administered decarboxylated cannabis pill. The pill includes decarboxylated cannabis in a gelatin capsule. The pill obviates smoking of cannabis for providing recreational and medicinal effects of cannabis. The pill is made by extracting cannabis extract from cannabis flowers, decarboxylating cannabis extract to release THC, and grinding down to powder for placing into a size 00 gelatin capsule for oral administration.

In this manner, the orally administered cannabis pill of the present invention accomplishes all of the foregoing objectives and provides users with cannabis pills made from decarboxylated cannabis ground down and put into gelatin capsules. The pill enables users to ingest cannabis without having to smoke or cat high calorie edibles and enables users to obtain pain and stress relief through cannabis with a simple pill. The pill is safe to consume, provides a desired or measured quantity of cannabis, and saves time and effort when using cannabis for medicinal and recreational purposes. The pill obviates smoking cannabis, eating edibles, and provides effective absorption of cannabis without affecting the lungs.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a method for producing an orally administered cannabis pill used for both medicinal and recreational purposes. The method comprising the steps of sourcing fresh and organic cannabis flowers containing cannabinoids, applying heat and pressure to the fresh cannabis flowers to extract essential oils and produce a concentrated cannabis extract in the form of a plurality of flat discs, decarboxylating the flat disc of cannabis extract to transform THCA into psychoactive THC (Δ9-THC), grinding the decarboxylated cannabis extract into a fine powder, and encapsulating the powdered cannabis extract into size 00 gelatin capsules, enabling for convenient and accurate dosing.

In a further embodiment, an orally administered decarboxylated cannabis pill is disclosed. The pill includes decarboxylated cannabis for medicinal and recreational purposes and consists of (i) 90-95 weight percentage decarboxylated cannabis extract powder, (ii) 3-5 weight percentage flavoring agent, (iii) 0.1-0.5 weight percentage mineral supplement, (iv) 0.1-0.2 weight percentage vitamin supplement, and (v) Lecithin.

In yet another embodiment, a capsule or pill for oral administration for providing cannabis is disclosed. The capsule includes 90-95% w/w decarboxylated cannabis extract powder, 3-5% w/w flavoring agent, 1-3% w/w Lecithin, and 1-3% w/w coconut oil, the capsule has the ability to digest inside the human body without affecting the lungs of a consumer of the capsule.

In yet another embodiment, the decarboxylation is performed by heating the cannabis extract between 100° C. and 123° C. for 10-15 minutes.

In another aspect, the cannabis pill has an average peak THC plasma level ranging from about 40 ng/ml to about 80 ng/ml.

In another embodiment, the cannabis flowers are selected from indica, sativa, and hybrid strains.

In yet another embodiment, a decarboxylated cannabis pill suitable for oral administration and designed for both medicinal and recreational purposes is disclosed. The pill has a size of 00 gelatin capsule for the convenient delivery of a suitable cannabis dose to consumers, wherein said cannabis pill is available in various container sizes, including 1 oz., 2 oz., 3 oz., 4 oz., or more, and is capable of exhibiting color changes over time due to its natural and organic ingredients.

In another aspect, the gelatin body can be made from cellulose or other plant-derived materials and are suitable for individuals who avoid animal products.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a flow chart depicting a process for making cannabis pills adapted for oral consumption in accordance with one embodiment of the present invention;

FIG. 2 illustrates a perspective view of one potential embodiment of the cannabis pill of the present invention in accordance with the disclosed structure; and

FIG. 3 illustrates an exemplary grinder used for manufacturing of the decarboxylated cannabis powder in accordance with one embodiment of the present invention.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for ingesting cannabis orally, typically in the form of pills such as capsules. Additionally, there is a long-felt need in the art for a cannabis pill that obviates smoking cannabis for recreational and medicinal benefits of cannabis. Moreover, there is a long-felt need in the art for an orally administered cannabis pill to help alleviate chronic pain and providing recreational effect of the cannabis. Further, there is a long-felt need in the art for a cannabis medication pill that uses gelatin capsules with decarboxylated cannabis for using cannabis for medicinal and recreational purposes. Furthermore, there is a long-felt need in the art for an oral cannabis pill that provides an adequate and precise dose of cannabis for medicinal or recreational purposes. Also, there is a long-felt need in the art for a cannabis pill that enables users to obtain pain and stress relief through cannabis while orally ingesting the pill without consuming high calorie edibles. Finally, there is a long-felt need in the art for a decarboxylated cannabis capsule or pill that provides optimal effect of cannabis without side effects of cannabis smoke and is safe for the lungs of users.

The present invention, in one exemplary embodiment, is a method for producing an orally administered cannabis pill used for both medicinal and recreational purposes. The method comprising the steps of sourcing fresh and organic cannabis flowers containing cannabinoids, applying heat and pressure to the fresh cannabis flowers to extract essential oils and produce a concentrated cannabis extract in the form of a plurality of flat discs, decarboxylating the flat disc of cannabis extract to transform THCA into psychoactive THC (Δ9-THC), grinding the decarboxylated cannabis extract into a fine powder, and encapsulating the powdered cannabis extract into size 00 gelatin capsules, enabling for convenient and accurate dosing.

Flavoring agents or flavonoids can be included in powder form in the pill for providing a flavor for easy ingestion. For enhancing the therapeutic properties of the pill, additional supplements including vitamin and mineral supplements can be added to the cannabis powder for the pills.

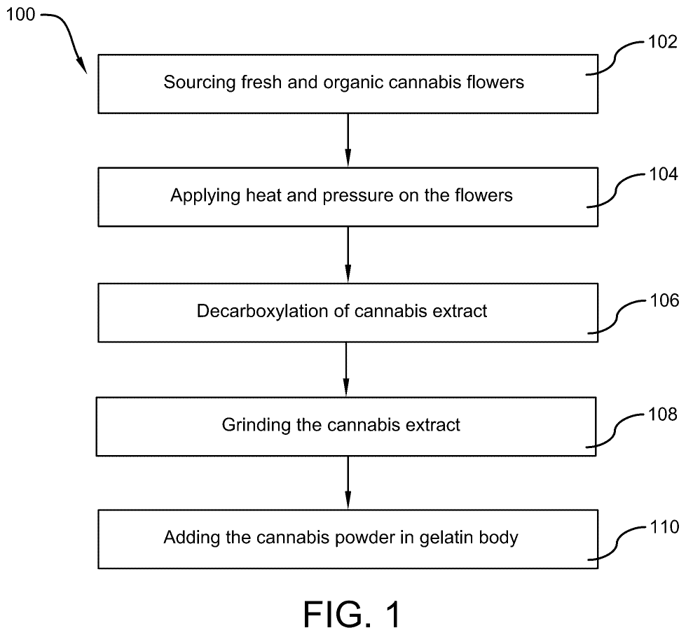

Referring initially to the drawings, FIG. 1 illustrates a flow chart depicting a process for making cannabis pills adapted for oral consumption in accordance with one embodiment of the present invention. The cannabis pill of the present invention is used for medicinal and recreational purposes and is easier to dose (i.e., measured quantities) compared to other forms of cannabis consumption like smoking or vaping. Initially, the process 100 of manufacturing the cannabis pills includes sourcing fresh and organic cannabis flowers (Step 102). The flowers can contain cannabinoids such as Tetrahydrocannabinol acid (THCA). The flowers can be dried flowers and can be one from indica, sativa, and hybrid. Also, the THC is largely concentrated around the flowering parts of the female plant.

In the next step 104, the fresh cannabis flowers are subjected to heat and pressure. More specifically, heat and pressure are applied to the cannabis flower to extract the cannabis oils from the flowers and to produce a concentrated form of cannabis extract which can be in the form of a flat disc. The pressure can be in the range of 4-10 bar and the temperature can be in the range of from about 40° C. to about 63° C. to convert all of the cannabis flowers into the cannabis extract. In one exemplary method, the heating is done by baking the cannabis flowers.

In the next step 106, a flat disc of the cannabis extract is subjected to further heating for decarboxylation. In the decarboxylation step, a carboxyl group from the THCA molecule is removed to transform the cannabis extract into psychoactive THC (Δ⁹-THC). Preferably, the cannabis extract is heated between 100° C. and 123° C. (212° F. and 253.4° F.) for 10-15 minutes and can be subjected to microwaves to avoid destroying THC. The decarboxylated cannabis contains from about 98% THC to about 99.5% THC. After the step of decarboxylation, in step 108, the activated cannabis extract is ground into a fine powder. A grinder can be used for grinding the cannabis extract for a uniform medicinal powder.

Finally, in step 110, the powder is then placed inside size 00 gelatin capsules. Size 00 offers a convenient dose for ingestion and enables for absorption of the THC and other cannabinoids through the digestive system and metabolized by the liver. The gelatin capsules are packed in containers for commercial distribution. The average peak THC plasma level of the capsule or pill can be from about 40 ng/ml to about 80 ng/ml and occurs within 10 minutes.

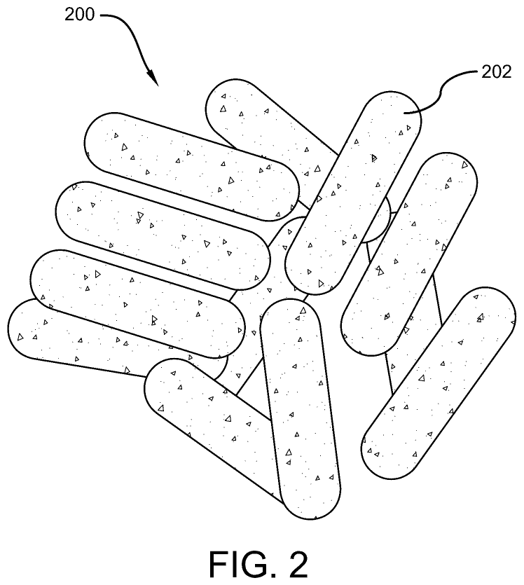

FIG. 2 illustrates a perspective view of one potential embodiment of the cannabis pill of the present invention in accordance with the disclosed structure. The decarboxylated cannabis pill 200 of the present invention is orally administered for medicinal and recreational purposes. The orally administered TCH pill 200 is preferably in the form of 00 gelatin capsule for providing an adequate and measured dose of cannabis to a consumer. The cannabis pills can be commercially available in small containers available in various sizes, such as, 1 oz., 2 oz., 3 oz., 4 oz., and more. The pill 200 can change color over time due to the natural and organic ingredients.

In some embodiments, the gelatin body 202 is discreet, odorless, and easy to transport, which can be beneficial for users who need to take cannabis medication without drawing attention. Further, the gelatin body 202 can be made from cellulose or other plant-derived materials and are suitable for individuals who avoid animal products. Alternatively, the gelatin body 202 can be made from animal-derived products. The pill 200 holds about 15 milligrams of cannabis powder but can vary depending on the density of the cannabis powder being encapsulated in the pill 200.

In some embodiments, a flavoring agent can be added to the cannabis extract. The flavor can be selected from a group of cocoa powder, mint extract, orange, or any other conventional natural or synthetic flavor. In some embodiments, the gelatin body 202 can be coated with the flavoring agent.

Table 1 below shows approximate concentrations of various ingredients in the decarboxylated cannabis pill 200.

Vitamin supplement can include Vitamin D which is essential for bone health and overall well-being. Vitamin C can be included which is an antioxidant that supports the immune system and aids in the absorption of non-heme iron. A comprehensive multivitamin supplement can be added to provide a balanced mix of vitamins and minerals to support overall health. Mineral supplement can include calcium, zinc, and iron for providing an overall health benefit to the consumers.

Lecithin is an emulsifier that can enhance the absorption of cannabinoids in the body. Coconut oil is added to improve the absorption of THC in the digestive system of a user.

TABLE 1

| | Composition in percentage by weight |
|---|---|
| Ingredients | (% w/w) |
| Decarboxylated cannabis | 90-95 |
| extract powder | |
| Flavoring agent | 3-5 |
| Mineral supplement | 0.1-0.5 |
| Vitamin supplement | 0.1-0.2 |
| Lecithin | 1-3 |

Table 2 below shows approximate concentrations of various ingredients in another decarboxylated cannabis pill 200.

TABLE 2

| | Composition in percentage by weight |
|---|---|
| Ingredients | (% w/w) |
| Decarboxylated cannabis | 90-95 |
| extract powder | |
| Flavoring agent | 3-5 |
| Lecithin | 1-3 |
| Coconut oil | 1-3 |

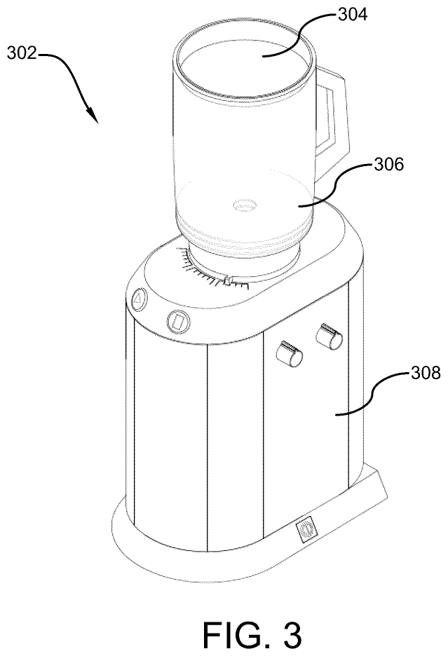

FIG. 3 illustrates an exemplary grinder used for manufacturing of the decarboxylated cannabis powder in accordance with one embodiment of the present invention. The grinder 302 can be designed for both commercial and personal use and can be used for grinding up to two kilograms of the cannabis extract. The grinder 302 includes a space 304 for storing and grinding cannabis extract discs 306 and a storage 308 for storing the powder for adding into gelatin body 202.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “decarboxylated cannabis pill”, “cannabis pill”, “pill”, and “orally administered THC pill” are interchangeable and refer to the orally administered decarboxylated cannabis pill 200 of the present invention.

Notwithstanding the foregoing, the orally administered decarboxylated cannabis pill 200 of the present invention can be of any suitable size and configuration as is known in the art without affecting the overall concept of the invention, provided that it accomplishes the above-stated objectives. One of ordinary skill in the art will appreciate the orally administered decarboxylated cannabis pill 200 as shown in the FIGS. is for illustrative purposes only, and that many other sizes and shapes of the orally administered decarboxylated cannabis pill 200 are well within the scope of the present disclosure. Although the dimensions of the orally administered decarboxylated cannabis pill 200 are important design parameters for user convenience, the orally administered decarboxylated cannabis pill 200 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A cannabis pill for medicinal and recreational consumption, the cannabis pill comprising:
a cannabis pill composition selected from the group consisting of a decarboxylated cannabis, a flavoring agent, a mineral supplement, a vitamin supplement, and a Lecithin, and combinations thereof.

2. The cannabis pill of claim 1, wherein said decarboxylated cannabis having a weight percent of said cannabis pill composition from 90% to 95%.

3. The cannabis pill composition of claim 2, wherein said flavoring agent having a weight percent of said cannabis pill composition from 3% to 5%.

4. The cannabis pill composition of claim 3, wherein said mineral supplement having a weight percent of said cannabis pill composition from 0.1% to 0.5%.

5. The cannabis pill composition of claim 4, wherein said vitamin supplement having a weight percent of said cannabis pill composition from 0.1% to 0.2%.

6. The cannabis pill composition of claim 5, wherein said Lecithin having a weight percent of said cannabis pill composition from 1% to 3%.

7. A cannabis capsule for medicinal and recreational consumption, the cannabis capsule comprising:
an encapsulated cannabis composition selected from the group consisting of a decarboxylated cannabis, a flavoring agent, a Lecithin, a coconut oil, and combinations thereof.

8. The cannabis capsule of claim 7, wherein said decarboxylated cannabis having a weight percent of said encapsulated cannabis composition from 90% to 95%.

9. The cannabis capsule composition of claim 8, wherein said flavoring agent having a weight percent of said encapsulated cannabis composition from 3% to 5%.

10. The cannabis capsule composition of claim 9, wherein said Lecithin having a weight percent of said encapsulated cannabis composition from 1% to 3%.

11. The cannabis capsule composition of claim 10, wherein said coconut oil having a weight percent of said encapsulated cannabis composition from 1% to 3%.

12. A method of making a cannabis capsule, the method comprising the steps of:
harvesting cannabis flowers;
drying said cannabis flowers;
applying a first heat and a first pressure to said cannabis flowers to remove cannabis oils from said cannabis flowers;
producing a concentrate of said cannabis oils;
applying a second heat to said concentrate of said cannabis oils to form a cannabis extract;
decarboxylating said cannabis extract to transform said cannabis extract into a psychoactive THC;
grinding said psychoactive THC into a cannabis powder; and
encapsulating said cannabis powder into gelatin capsules.

13. The method of making a cannabis capsule of claim 12, wherein said psychoactive THC having a concentration from 98% THC to 99.5% THC.

14. The method of making a cannabis capsule of claim 12 further comprising a step of coating said gelatin capsules with a flavoring agent.

15. The method of making a cannabis capsule of claim 12, wherein said second heat is from 100° C. to 123° C.

16. The method of making a cannabis capsule of claim 15, wherein a duration of said second heat is from 10 minutes to 15 minutes.

17. The method of making a cannabis capsule of claim 16, wherein said second heat is applied by microwaving.

18. The method of making a cannabis capsule of claim 12, wherein said cannabis extract having Tetrahydrocannabinol acid.

19. The method of making a cannabis capsule of claim 12, wherein said cannabis flowers selected from the group consisting of an indica and a sativa.

20. The method of making a cannabis capsule of claim 12, wherein said first heat including a temperature from 40° C. to 63° C., and further wherein said first pressure is from 4 bar to 10 bar.
